(19) United States
(12) Patent Application Publication
(10) Pub. No.: US 2022/0226140 A1
(43) Pub. Date: Jul. 21, 2022

(54) BACK BRACE, SYSTEM FOR USE WITH BACK BRACE, AND METHOD FOR EFFICIENT MANAGEMENT OF SPINE DEFORMATION TREATMENT

(71) Applicant: AIDPLEX INC., LEWES, DE (US)

(72) Inventors: CHARALAMPOS GEREMTZES, EDESSA (GR); DIMITRIOS MOUSTAKAS, RODOS (GR); ANARGYROS MOURATIDIS, DRAMA (GR); ANASTASIOS ANASTASIADIS, EDESSA (GR); KONSTANTINOS PARASKEVAS, EDESSA (GR)

(73) Assignee: AIDPLEX INC., LEWES, DE (US)

(21) Appl. No.: 17/575,736

(22) Filed: Jan. 14, 2022

Related U.S. Application Data
(60) Provisional application No. 63/137,765, filed on Jan. 15, 2021.

(51) Int. Cl. A61F 5/02; A61B 5/00; A61B 5/103

(52) U.S. Cl. CPC A61F 5/024; A61B 2562/0247; A61B 5/0004; A61B 5/0022; A61B 5/0024; A61B 5/0053; A61B 5/1036; A61B 5/4566; A61B 5/6823; A61B 5/6831; A61B 5/6885; A61B 5/7275; A61B 5/742; A61B 5/7465; A61B 5/0082; A61B 5/4561; A61B 5/6805; A61B 5/6812; A61B 2505/07; A61F 5/022

(57) ABSTRACT

A back brace is configured to be worn by a patient for treating spinal deformations. The back brace has at least one pressure sensor configured to be attached to the back brace at a point of pressure with the body of a patient, and at least one strap configured to be tightened for adjusting a pressure at the point of pressure. A control unit is configured to be attached to one of an outside of the back brace and inside a cavity formed in the back brace. The control unit is configured for (a) processing signals from the at least one pressure sensor, (b) communicating with at least one external computing apparatus, (c) informing and guiding in real time and incentivizing a patient to more efficiently wear the back brace by at least providing indications to the patient for tightening the at least one strap by comparing a pressure value provided by the at least one pressure sensor with at least one calibration value, and (d) enabling a doctor to more efficiently monitor, intervene, and manage the patient and treatment in real time.

### RELATED APPLICATION

This application claims the benefit of U.S. Provisional Patent Application No. 63/137,765 filed on Jan. 15, 2021.

### BACKGROUND

### Field

The present invention relates generally to techniques for efficiently managing spine deformation treatment, and more particularly to a back brace, a system and method for efficiently managing spine deformation treatment.

### Background Information

Scoliosis, one type of spine deformation, affects 2-3 percent of the population, or an estimated six to nine million people in the United States. Scoliosis can develop in infancy or early childhood. However, the primary age of onset for scoliosis is 10-15 years old, occurring equally among both genders. Females are eight times more likely to progress to a curve magnitude that requires treatment. Every year, scoliosis patients make more than 600,000 visits to private doctor's offices, an estimated 30,000 children are fitted with a back brace and 38,000 patients undergo spinal fusion surgery.

Back braces are only effective in patients who have not reached skeletal maturity. If the child is still growing and his or her curve is between 25 degrees and 40 degrees, a brace may be recommended to prevent the curve from progressing. There have been improvements in brace design and the newer models fit under the arm, not around the neck. There are several different types of braces available. While there is some disagreement among experts as to which type of brace is most effective, large studies indicate that braces, when used with full compliance, successfully stop curve progression in about 80 percent of children with scoliosis. For optimal effectiveness, the brace should be checked regularly to assure a proper fit and may need to be worn 16 to 23 hours every day until growth stops.

A scoliosis curve that is 50 degrees by the time an adolescent reaches skeletal maturity (about the age of 14 or 15 for girls and 16 or 17 for boys) will continue to progress throughout adulthood. These types of curves are likely to become a severe deformity that requires surgery. Therefore, the goal of bracing is to avoid a major surgery by either stopping curve progression altogether or at least preventing it from reaching 40 or 50 degrees at the time of skeletal maturity.

Having scoliosis can be emotionally upsetting for adolescents. Scoliosis can cause adolescents stress and negatively affect their body image.

Bracing is currently the primary method for treating moderate idiopathic scoliosis (IS) during the developmental phase of growth. Accurate evaluation of patient compliance with scoliosis brace usage has been a challenge for doctors treating patients with IS. This inability to accurately measure compliance has resulted in difficulty in determining brace treatment efficacy. Previous studies have used either questionnaires to determine compliance or verbal reports on the number of hours worn to a nurse at the clinic. The number of wear hours reported in these studies was subjective and difficult to verify due to reliance on patient recall, with the possibility of false reporting. Monitoring devices were proposed that can address some of the earlier technical shortcomings and to determine the adequacy of using these devices as a reliable and accurate means of measuring the compliance of brace wear in the treatment of idiopathic scoliosis. For example, systems are known in the prior art which measure the number of hours a brace is worn by a patient using thermal and/or pressure sensors. Studies have shown that monitoring of the patient's compliance has affected positively the wearing time of the back brace.

However, recording the daily usage of the brace is not enough for monitoring a patient's progress in the treatment as the previous systems cannot measure the improvement of the patient's spine curvatures as a result of the use of the brace in the treatment. For this reason, it is not possible for such systems to provide guidance to the patient, or adapt the treatment regime.

Also, patients, especially those of younger ages, easily lose motivation to follow their treatment regime or cut corners to make their lives with the brace easier. In addition, patients have no guidance on how to correctly and efficiently use the brace, until their next appointment with their doctor. As these appointments are usually scheduled every few months, patients loose motivation and/or have no means to correct mistakes (e.g. improper fitting on the torso, or improper tightening of the fitting straps) in using the brace in the prescribed, correct way.

Despite the existence of electronic monitoring systems fitted on braces (e.g. heat and/or pressure sensors for detecting wear time of the brace), monitoring a patient's progress needs to be done by a doctor. However, this is a rather inefficient process due to rare appointments with the doctor. The clinician must determine the progress of the treatment using the patient's curve and adjust the brace accordingly. The number of wear hours reported is subjective and difficult to verify due to reliance on patient recall, with the possibility of false reporting. Also, despite being accurate, the sensory data for the duration of use of the brace, provided by the above monitoring systems is not sufficient for estimating important parameters used in judging correct use of the brace according to the prescribed treatment regime. For example, the fact that the user wears the brace for the prescribed number of hours does not guarantee that the brace is worn properly according to the doctor's instructions. Low (or some cases excessive) pressure may be applied by the brace to pressure points on the patient's spine, or even be applied at wrong pressure points because the brace is not correctly positioned, or in more extreme situations due to problems in the 3D shape of the brace, failure of the brace due to wear or improper use, or simply due to changes in the patient's body as a result of putting on or losing weight, or developing extra muscle mass due to exercising.

Without a way to access such data for evaluating the compliance of the patient to the doctor's instructions, the doctor has to physically examine the patient for evaluating the patient's progress in the treatment (e.g. how well his spine reacts). This means that the doctor will schedule an appointment every few months without having any way to check progress in the meantime. This is standard practice, which may, however, seriously affect patient progress in the treatment as for situations of no conformance to the regime, problems with brace itself, or unexpected response of the patient's spine to the treatment, the doctor can assess the situation and make adjustments only during the physical examination. In other words, months may be lost, seriously setting back the patient in his treatment, or even aggravating the spinal deformation.

There is, therefore, the problem of facilitating doctors in efficiently monitoring and managing treatment of spinal deformations, while aiding and incentivizing and guiding patients to efficiently follow the prescribed treatment regime.

### SUMMARY

The present invention solves the problem of facilitating doctors in efficiently monitoring and managing treatment of spinal deformations, while aiding and incentivizing and guiding patients to efficiently follow the prescribed treatment regime. The invention solves the problem using real-time pressure measurements to calculate the pressure applied at selected pressure points on the patient's spine, data analysis, and scheduling of doctor interventions and appointments with the patient for optimizing the personalized treatment of the patient's spinal deformation (such as scoliosis).

A system is presented which uses pressure sensors attached on the inner surface of a brace for detecting when the brace is worn by the patient and the pressures applied by the brace at pre-determined pressure points onto the patient's torso. These points are selected by the doctor to apply pressures on the spine for forcing it to acquire its physiological curvatures and are implemented by 3D modeling of the brace to the 3D model of the patient's torso and the 3D deformations of his spine. The pressure measurements are collected and time-stamped by a wearable device wired or wirelessly connected to the sensors. The wearable device locally stores the time-stamped pressure measurements in real time and transmits them to an application running at a smartphone or other computing device used by the patient and/or his family members in case of a child patient. The patient's smartphone connects to a server and uploads the data. The server processes the data and creates metrics relating to the usage of the brace. The metrics are associated by the server with a personalized treatment regime created by the patient's doctor, using his own computing device, and communicated to the server. Both the patient and the doctor can access the processed data on the server through the applications running at the respective devices. Different views and tools for the same data are offered to the patient and his doctor in order to serve the needs of each one.

The patient can access from the server the metrics in the form of a graphical presentation, instruction on how to efficiently use the brace, advice from the doctor for correcting the application of the brace, changes in the treatment regime, and a gamification of the treatment for incentivizing him to follow the treatment. The gamification is particularly suitable to children at preadolescent age, who are presented with a video game or a gamified application, which is adapted to the patient's condition and/or preferences and uses awards related to the correct use of the brace according to the prescribed treatment regime and patient progress.

The doctor can create on the server a patient account and treatment regime, access from the server the metrics, adapt the treatment regime, send advice to the patient for correcting the application of the brace, schedule appointments with the patient at optimal times and dates that are beneficial for managing the progress of the therapy based on the metrics (e.g. for minimizing the need to order and examine a spine X-ray), and receive alerts associated with the metrics and the patient's progress.

The system runs software to implement a method along the lines described above for the system components (i.e. wearable system and sensors, patient's mobile application, server, and doctors web application) and stakeholders (i.e. the patients, and the doctor).

Beyond accessing the above needs of the patient and doctor, the system also innovates by considering both the patient's and the clinician's perspective, providing real-time monitoring and management, and addressing the patient's emotional state and needs by providing gamification and real-time metrics and advice aiming to motivate the patient to wear the brace for the prescribed time and in the correct manner.

A back brace is also presented, having one or more straps for tightening in order to adjust the pressure at each of one or more points of pressure, one pressure sensor attached to the inner side of the brace at each of the one or more points of pressure for sensing the pressure exerted to the wearer of the brace, and a wearable device connected to the sensors. The wearable device may be attached to the outside of the brace or inside a cavity formed in the material of the brace. The wearable device processes signal from the pressure sensors, communicates with a patient's computing apparatus, and informs and guides in real time, and incentivizes the patient to more efficiently wear the back brace, by at least providing indications to the patient for tightening the at least one strap by comparing a pressure value provided by each of the at least one pressure sensor with at least one calibration value, and enables a doctor to more efficiently monitor, intervene, and manage a plurality of patients and their treatments in real time.

### BRIEF DESCRIPTION OF THE DRAWINGS

FIG. 1A shows a pressure sensor installed at the inner side of a back brace and a control unit installed on the outside of the same back brace according to the present invention.

FIG. 1B shows a pressure sensor installed at the inner side of a back brace and a control unit installed in a cavity in the same back brace according to the present invention.

FIG. 2A shows a blown-up side view of a pressure sensor assembly.

FIG. 2B shows a side view of a pressure sensor assembly.

FIG. 3A shows a top-down view of a rectangular pressure sensor.

FIG. 3B shows a bottom-up view of a rectangular pressure sensor.

FIG. 4A shows a top-down view of a round pressure sensor.

FIG. 4B shows a bottom-up view of a round pressure sensor.

FIG. 5 shows a high-level block diagram of a control unit.

FIG. 6A shows a perspective view of the main body of a casing for the control unit.

FIG. 6B shows a perspective view of the cover of the casing for the control unit.

FIG. 7 shows a high-level flow diagram of the operation of a system according to the present invention.

FIG. 8 shows a flow diagram of the first setup of the system.

FIG. 9 shows a flow diagram of installing the wearable device.

FIG. 10 shows a flow diagram of connecting the wearable device with a mobile application.

FIG. 11 shows a flow diagram of calibrating the system.

FIG. 12 shows a flow diagram of adjusting the system.

FIG. 13 shows a schematic diagram of the interaction of the system hardware.

### DETAILED DESCRIPTION

The word “Any embodiment described herein as “exemplary” is not necessarily to be construed as preferred or advantageous over other embodiments.

The term “exemplary” is used herein to mean “serving as an example, instance, or illustration”.

The acronym “A/D” is intended to mean “Analogue to Digital”.

The acronym “App” is intended to mean “Application”.

The acronym “ASCII” is intended to mean “American Standard Code for Information Interchange”.

The acronym “ASIC” is intended to mean “Application-Specific Integrated Circuit”.

The acronym “Bluetooth LE” is intended to mean “Bluetooth Low Energy”.

The acronym “CD” is intended to mean “Compact Disc”.

The acronym “DSL” is intended to mean “Digital Subscriber Line”.

The acronym “DVD” is intended to mean “Digital Versatile Disc”.

The acronym “HIS” is intended to mean “Hospital Information System”.

The acronym “IS” is intended to mean “Idiopathic Scoliosis”.

The acronym “IMU” is intended to mean “Inertial Measurement Unit”.

The acronym “LED” is intended to mean “Light Emitting Diode”.

The acronym “Mob” is intended to mean “Mobile”.

The acronym “PACS” is intended to mean “Patient Archiving and Communication System”.

The acronym “PIF” is intended to mean “Patient Information Folder”.

The acronym “QR” is intended to mean “Quick Response”.

The acronym “RTC” is intended to mean “Real Time Clock”.

The acronym “XML” is intended to mean “eXtensible Markup Language”.

The term “mobile device” may be used interchangeably with “client device” and “portable device with wireless capabilities”.

The term “user” may be used interchangeably with “regular user”, “ordinary user”, and “client”. It may also be used to mean “user of an application” or “user of a service”. It may also be used to refer to a “patient using a device, application, or service”, or to a “doctor using a device, application, or service”, unless otherwise explicitly stated or implicitly hinted at in the description, or obvious to a reader of ordinary skill in related art that these terms refer to different things, as this is apparent by the context of the discussion in which they appear.

The term “physician” may be used interchangeably with “doctor”.

The term “system” may be used interchangeably with “device”, “computing device”, “apparatus”, “computing apparatus”, and “service”, except where it is obvious to a reader of ordinary skill in related art that these terms refer to different things, as this is apparent by the context of the discussion in which they appear. Under any circumstance, and unless otherwise explicitly stated or implicitly hinted at in the description, these six terms should be considered to have the broadest meaning i.e. that of encompassing all six.

The term “module” may be used interchangeably with “unit” or “subunit”, except where it is obvious to a reader of ordinary skill in related art that these terms refer to different things, as this is apparent by the context of the discussion in which they appear.

The term “back brace” may be used interchangeably with “brace”.

The term “treating spinal deformations” may be interpreted as “constraining the spine” or “correcting spinal deformations”, as this is apparent by the context of the discussion in which they appear.

A Back Brace Equipped with the Hardware of the Present Invention

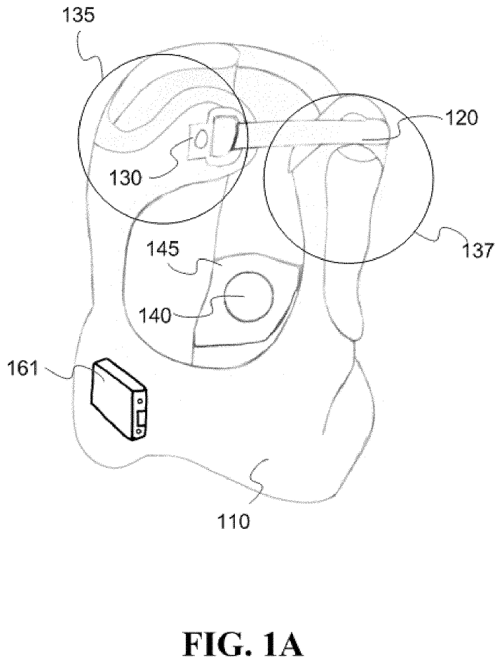

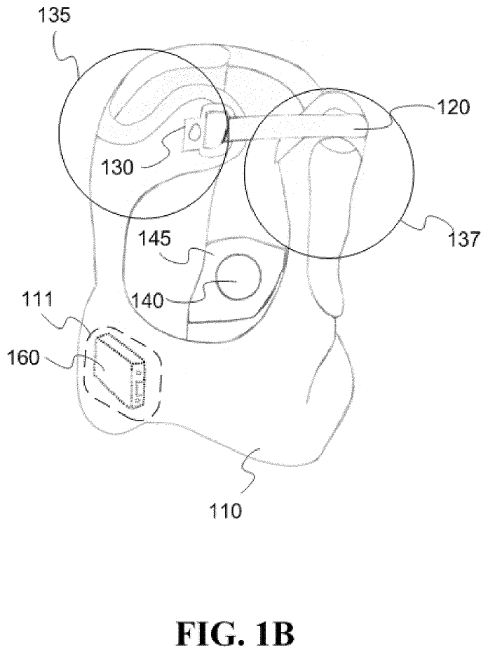

FIG. 1A shows a pressure sensor installed at the inner side of a back brace and a control unit 161 (e.g., wearable device) installed on the outside of the same back brace according to the present invention. A back brace 110, designed to fit on the 3D shape of a patient's torso and to apply adjustable pressures at selected pressure points for treating, or correcting spinal deformation and/or constraining the spine, is equipped with one or more straps 120. Strap 120 is used to tighten the back brace at predefined locations, for adjusting the pressures at the selected pressure points. In an effort to better control the pressures exerted at the pressure points, additional straps may be used (not shown). Strap 120 is typically fixed at or passes through a hook 130 attached at portion 135 of the back brace 110, and attaches at one end at a portion 137 of the back brace 110. The function of the strap 120 is to pull portion 135 close to portion 137 of the back brace 110, and vice versa. Strap 120 is typically equipped at one side with a securing device, like a Velcro® tape dimensioned to securely attach to a matching Velcro® tape attached at portion 137. In alternative exemplary implementations, other means of attaching the strap 120 to portion 137 may be used, and a system not using Velcro® tapes may be selected. Such choices are well known in the prior art.

Back brace 110 is equipped on its inner surface with one or more pressure sensors which, are securely attached to the back brace with an adhesive, at an area of contact 145 with the human torso. Area of contact 145 contains the point of pressure to be applied to the corresponding area of the patient's torso for correcting a spinal deformation and/or constraining the spine.

The pressure sensor is covered with a foam material 140, such as foam strips, which is intended to make the sensor comfortable for the patient wearing the brace 110. The pressure sensors can be placed between the inner surface of the back brace 110 and the foam strip that the patient's doctor attaches inside the brace, at the pressure points of the brace.

The pressure sensor(s) are connected to wearable device 161 which collects signals from the pressure sensor(s). Wearable device 161 is typically attached on the outside of the brace, in a manner that allows easy detachment and reattachment. Usually, the wearable device 161 is attached by Velcro® tape, at any point on the outside of the back brace (more usually at the side at waist level), or it is attached at a strap 120. In alternative exemplary embodiments, the wearable device 161 may be worn by the patient on a belt, wrist-band or the like, or carried into a pocket.

The connection of the pressure sensors with the wearable device is typically via cables attached (e.g., with a self-adhesive tape, or other known attachment mechanism) to the back brace. In alternative exemplary implementations, the connection is wireless, avoiding the need for cables. If such a wireless connection is chosen, the pressure sensors must be selected from pressure sensors with integrated wireless capabilities or they should be connected to wireless transceivers at a close distance to or in contact with a side face of the sensors, again for avoiding using cables.

FIG. 1B shows another embodiment of the present invention in which the pressure sensor is installed at the inner side of back brace 110 and a control unit (denoted with reference number 160), such as a wearable device, is installed in a cavity 111 in the same back brace. FIG. 1B is identical to FIG. 1A except that the control unit 160 is installed in the cavity 111 formed inside the material forming the back brace 110. Braces are usually made from polypropylene or polyethylene which are thermoformable materials so by applying heat to the desired location of the brace, the brace manufacturer can create a cavity 111 to fit the wearable device 160. Also, some holes for cables, such as for the control unit 160, are opened either with a drill or by heat treatment of the brace material.

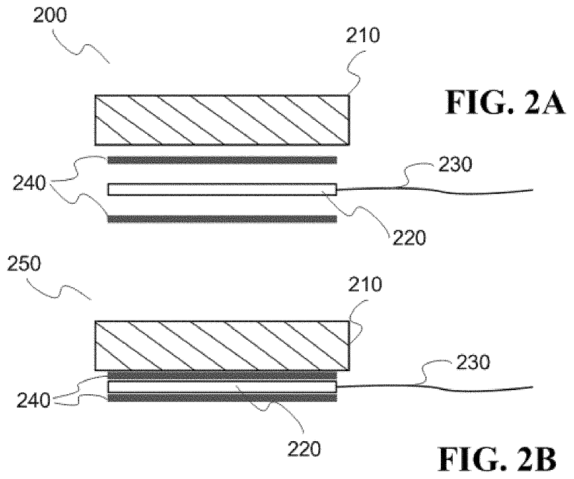

FIG. 2A shows a blown-up side view of a pressure sensor assembly. Pressure sensor assembly 200 comprises a flat sensor 220 sandwiched between two adhesive surfaces 240 running along the two parallel flat surfaces of the sensor 220. On one side of the pressure sensor 220, the adhesive surface 240 attaches the sensor to foam material 210, and on the opposite flat surface of the sensor 220, adhesive 240 attaches the sensor to the inner surface of the back brace 110. In the illustrated example, sensor 220 is connected with a cable 230 which is intended for connecting the sensor 220 to wearable device 160, or to a wireless transceiver (not shown) close or at contact with the sensor 220.

Sensor 220 may be selected from known resistive or capacitive pressure sensors. In alternative implementations, other types of pressure sensors may be used, without limiting the scope of protection of the present invention.

FIG. 2B shows a side view of a pressure sensor assembly. Pressure sensor assembly 250 comprises a flat sensor 220 sandwiched between two adhesive surfaces 240 running along the two parallel flat surfaces of the sensor 220. On one side of the pressure sensor 220, the adhesive surface 240 attaches the sensor to foam material 210, and on the opposite flat surface of the sensor 220, adhesive 240 attaches the sensor to the inner surface of the back brace 110. In the illustrated example, sensor 220 is connected with a cable 230.

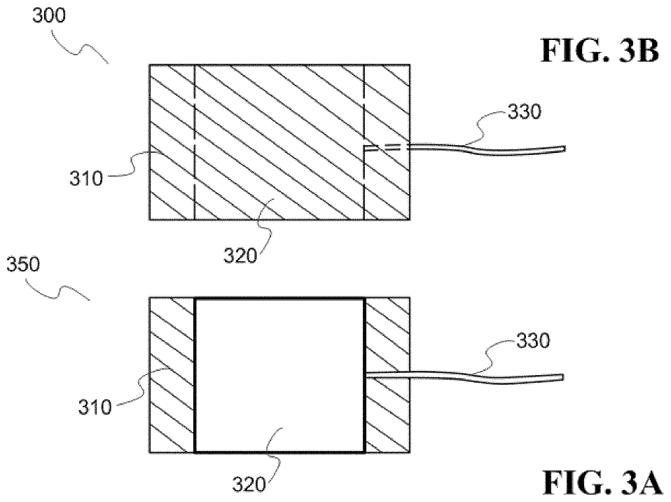

FIG. 3A shows a top-down view of a rectangular pressure sensor. In the top-down view 300, on the rectangular pressure sensor 320 is attached foam material 310. For extra comfort, the foam material 310 is optionally chosen to completely cover and extend beyond the area of the pressure sensor. Sensor 320 is connected with cable 330.

FIG. 3B shows a bottom-up view of a rectangular pressure sensor. In the bottom-up view 300, under the rectangular pressure sensor 320 is attached foam material 310. For extra comfort, the foam material 310 is optionally chosen to completely cover and extend beyond the area of the pressure sensor. Sensor 320 is connected with cable 330.

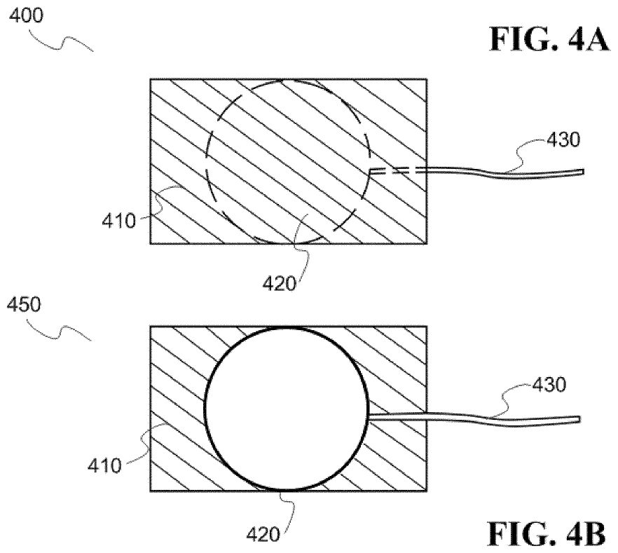

FIG. 4A shows a top-down view of a round pressure sensor. In the top-down view 400, on the round pressure sensor 420 is attached foam material 410. For extra comfort, the foam material 410 is optionally chosen to completely cover and extend beyond the area of the pressure sensor. Sensor 420 is connected with cable 430.

FIG. 4B shows a bottom-up view of a round pressure sensor. In the bottom-up view 400, under the round pressure sensor 420 is attached foam material 410. For extra comfort, the foam material 410 is optionally chosen to completely cover and extend beyond the area of the pressure sensor. Sensor 420 is connected with cable 430.

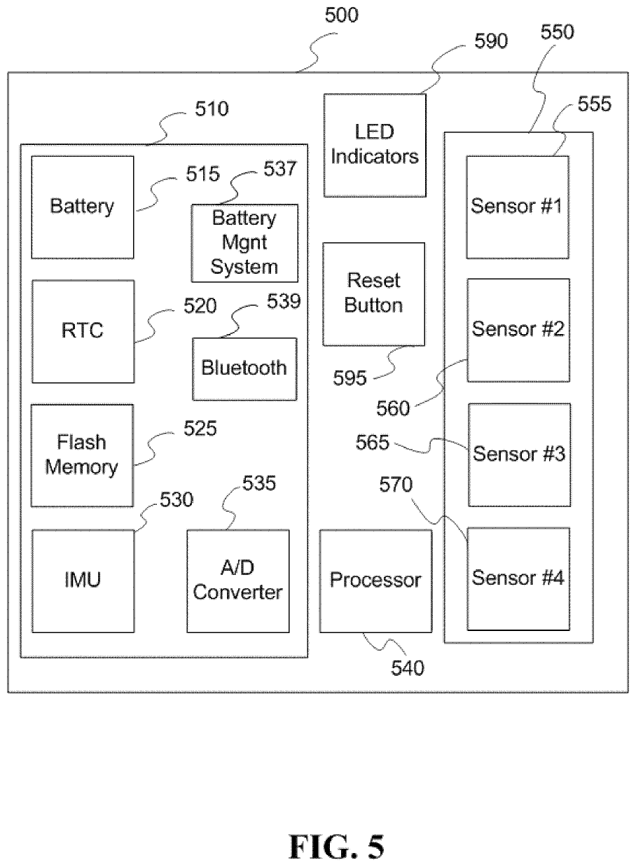

FIG. 5 shows a high-level block diagram of a control unit. Control unit 500 may be in the form of a wearable device and contains a processor 540, support modules 510, a reset/power button 595, and, optionally, light indications 590.

Microprocessor 540 may be selected from any known embedded microprocessors, Application-Specific Integrated Circuit (ASICS), and the like, etc.

Support modules 510, contain a battery 515 for powering control unit 500, a Real Time Clock (RTC) 520 for providing time-date data to processor 540, a flash memory 525 for storing pressure measurement from pressure sensors 220 and computer instructions, an Inertial Measurement Unit (IMU) 530 for detecting patient torso's motion, orientation and acceleration, an Analogue to Digital (A/D) converter 535 for converting analogue signals from pressure sensors 220 to digital signals fed to processor 540, a Bluetooth module 539 for wirelessly communicating with external computing apparatuses that receive sensory data associated with the wearing of the back brace, and battery management system 537 for managing battery charging and power use.

Flash memory 525 may be replaced or supplemented with other types of known memory modules. IMU 530 may be implemented with an accelerometer, inertial sensor, and a magnetometer, known in the prior art.

Pressure sensors 220 may be of any number matching the number of pressure points a doctor may select and connect to processor 540 via A/D converter 535.

In an alternative exemplary embodiment, pressure sensors 550 (in the illustrated example four pressure sensors 555, 560, 565, 570 are shown which may be of the same or different types) are part of control unit 500.

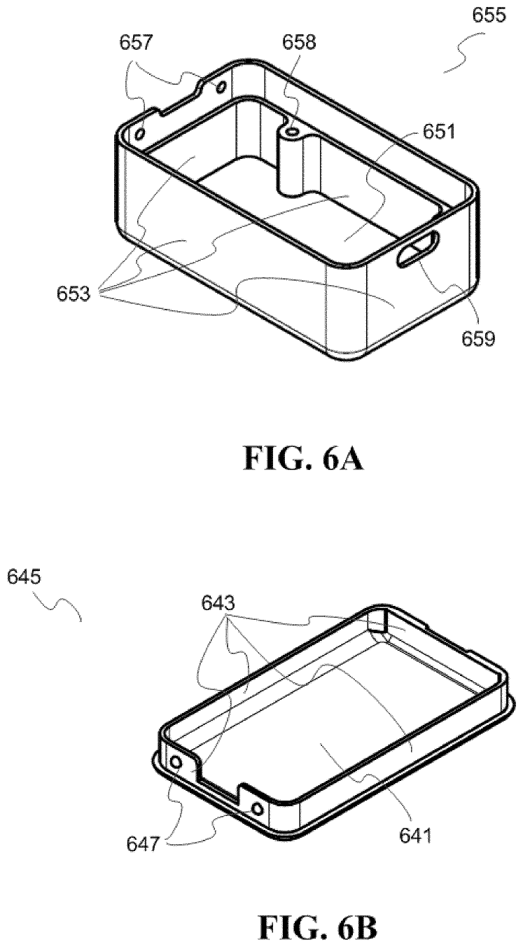

FIG. 6A shows a perspective view of the main body of a casing for the control unit. The main body of casing 655 is made of five flat surfaces forming a box open at one side. The dimensions of the box are selected to contain control unit 500. The bottom side 651 (as shown in FIG. 6A) of the main body of casing 655 has a surface area considerably larger than the other four sides 653, which are attached to the bottom side 651 at about 90° and are positioned at about 90° relative to each other. This exemplary design may be modified up to the extent that the four sides 653 have a profile that is suitably low for not protruding excessively out of its point of attachment on the external surface of the back brace, or the point of attachment on the patient's body, or the pocket it is carried in, as can easily be appreciated by a person skilled in related art. For extra comfort the edges where sides 651, 653 are attached to each other are rounded. The main body of casing 655 also has features 657, which form part of a snap fit mechanism for securely attaching a cover to the main body of casing 655, feature 658 which is a protrusion for screwing the control unit 500 on the main body of casing 655, and feature 659 which is an opening for cables connecting control unit 500 (which is placed in the inside of the main body of casing 655), with pressure sensors 220 or 550. In alternative exemplary embodiments other attachment mechanisms, known in the art, may be used for the cover and the control unit 500.

FIG. 6B shows a perspective view of the cover of the casing for the control unit. The cover of casing 645 is made of five flat surfaces forming a shallow box open at one side. The dimensions of the shallow box are selected to fit with the main body of casing 655. The upper side 641 (as shown in FIG. 6B) of the cover of casing 645 has a surface area considerably larger than the other four sides 643, which are attached to the bottom side 641 at about 90° and are positioned at about 90° relative to each other. This exemplary design may be modified up to the extent that the four sides 643 have a profile that is suitably low for not protruding excessively out of its point of attachment on the external surface of the back brace, or the point of attachment on the patient's body, or the pocket it is carried in. For extra comfort the edges where sides 641, 643 are attached to each other are rounded. The cover of casing 645 also has features 647, which form part of the snap fit mechanism for securely attaching the cover of casing 645 on the main body of casing 655. In alternative exemplary embodiments other attachment mechanisms, known in the art, may be used for the cover and the control unit 500. The dimensions of cover 645 closely match the dimensions of main body of casing 655, so as to allow cover 645 and main body of casing 655 to be joined into a robust case, forming wearable device 160, for housing control unit 500. The resulting case 160 may optionally be made water resistant or water tight for extra protection of the electronics of control unit 500.

System Operation

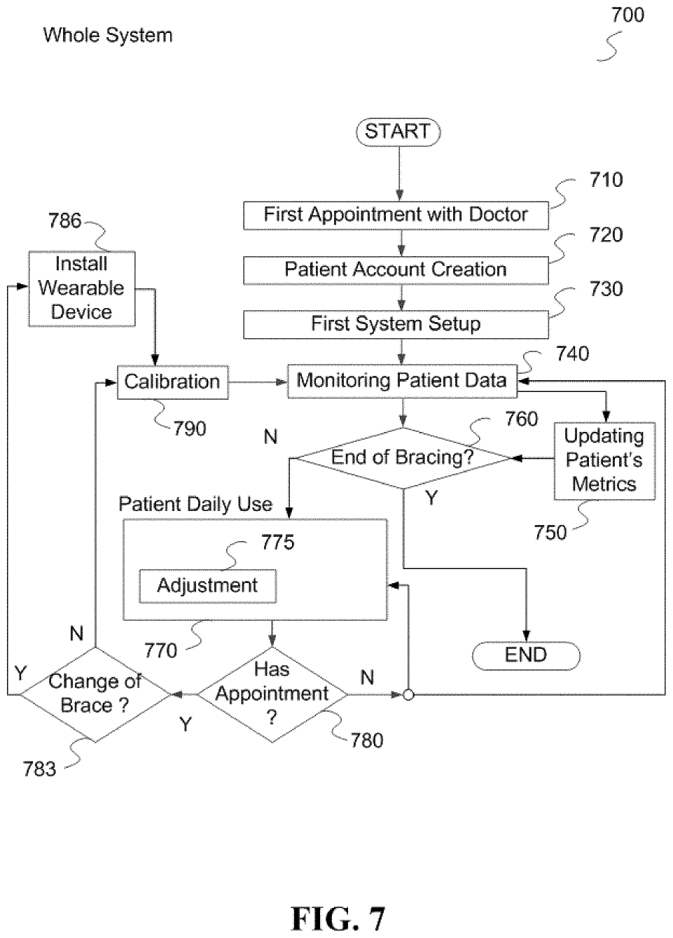

FIG. 7 shows a high-level flow diagram of the operation of a system according to the present invention.

Operation flow diagram 700 starts with the patient attending a first appointment for examination with his doctor 710. The doctor creates a patient account 720 using his computing apparatus (e.g. a smartphone, or any other computing apparatus), by connecting to a server.

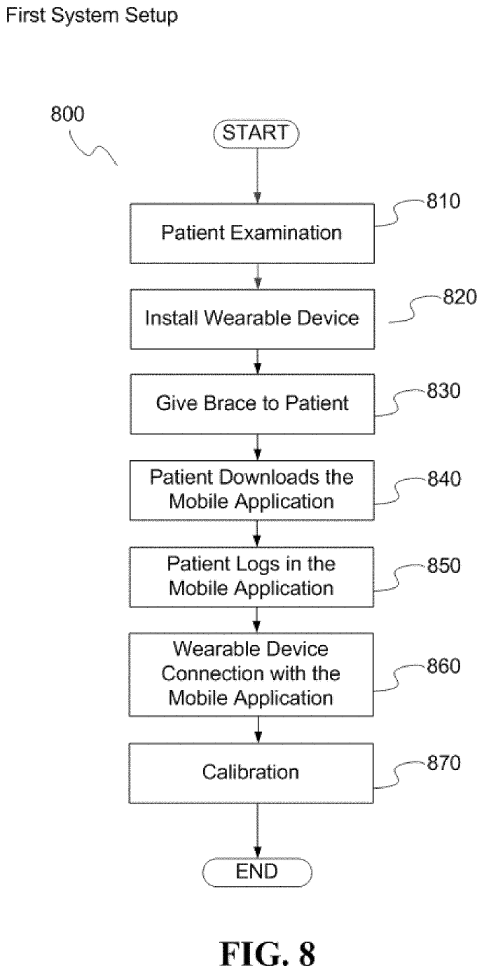

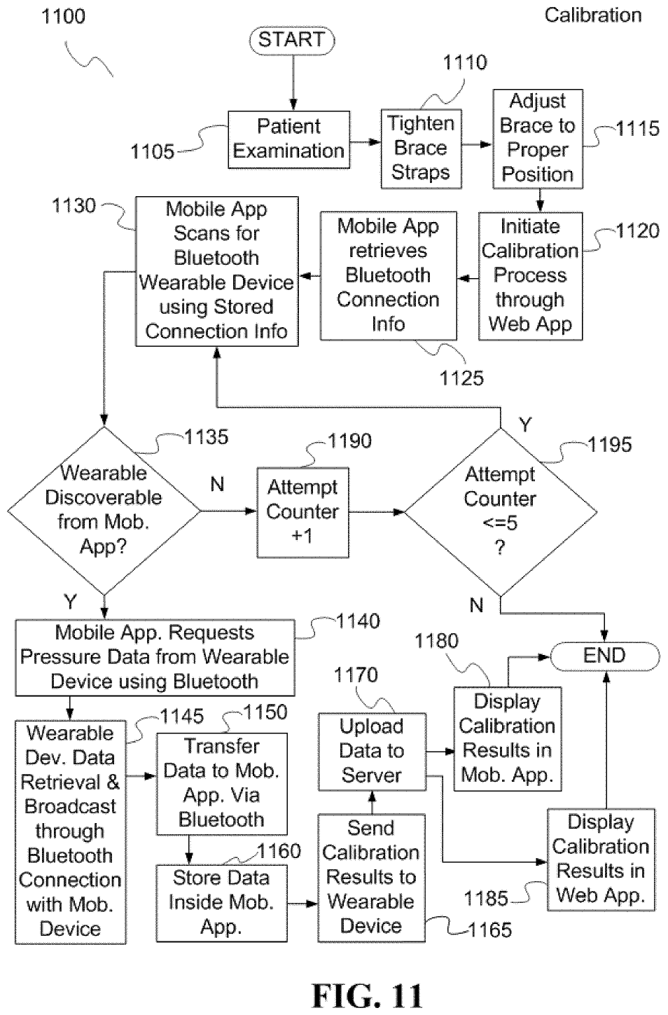

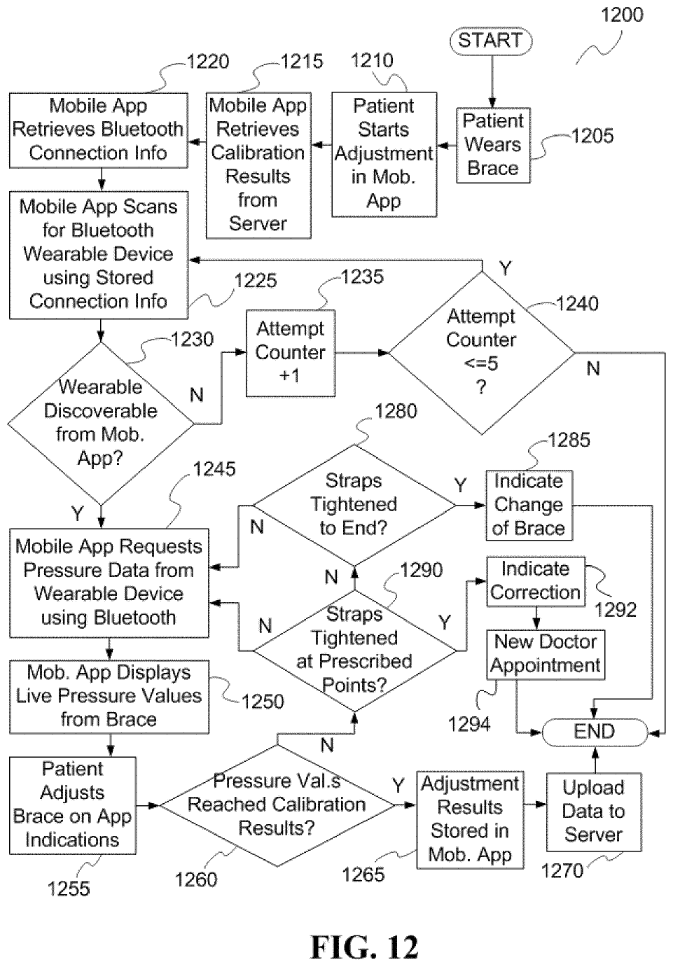

A first system setup 730 is done (please refer to FIG. 8) and the system monitors patient data 740 at the server and updates patient's metrics 750 at the server. If the bracing period is over 760, operation flow diagram 700 ends. Otherwise, operation flow diagram 700 continues with patient daily use 770, which includes an adjustment step 775 (please refer to FIG. 12). Patient daily use 770 continues until an appointment with the doctor is attended by the patient 780, when the system checks for a change in the back brace 783. If a change is detected, a wearable device is installed 786 and calibration is executed 790 (please refer to FIG. 11), followed by the monitoring patient data step 740. If no brace change is detected 783, the calibration step 790 is executed.

First System Setup

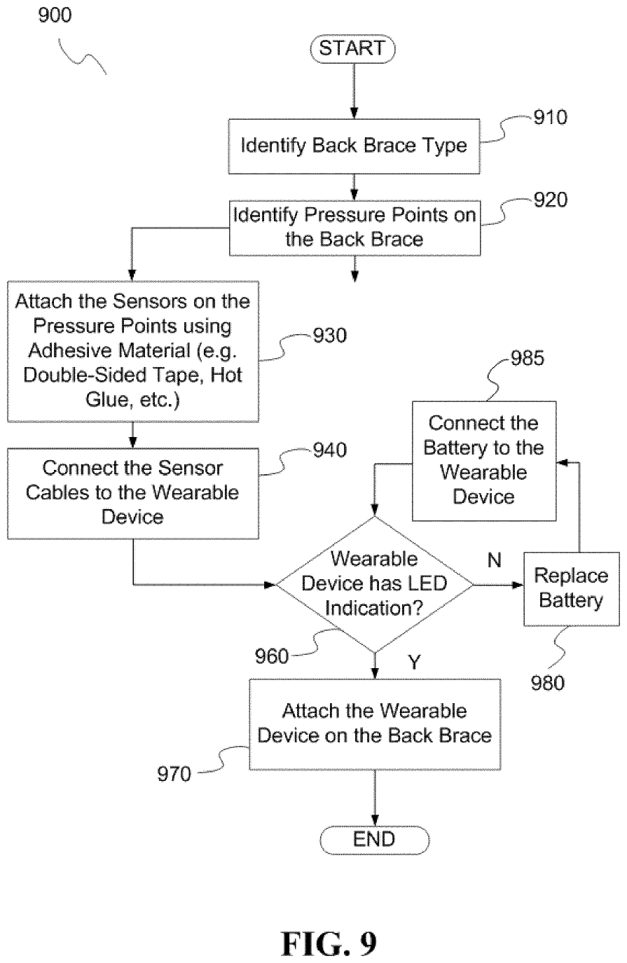

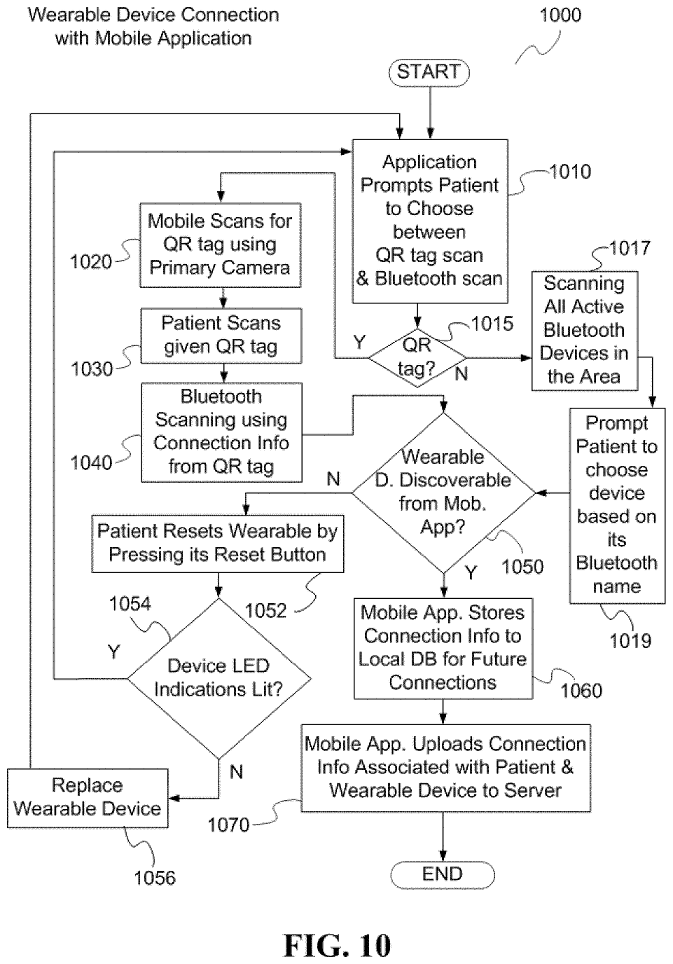

FIG. 8 shows a flow diagram of the first setup of the system. Flowchart 800 starts with a doctor examining the patient 810 during an appointment and installing 820 the wearable device 160, 500 and pressure sensors 220 or 550 to back brace 110 (please refer to FIG. 9). Having installed the wearable device, the doctor gives the brace to his patient 830. The patient downloads a mobile application 840 to his smartphone (or any computing apparatus) and logs-in the application 850. Wearable device 160, 500 then connects with the mobile application 860 (please refer to FIG. 10) and the system enters calibration step 870 before the end of the first system setup 800.

Installing the Wearable Device

FIG. 9 shows a flow diagram of installing the wearable device. Flowchart 900 starts with the doctor identifying the type of back brace 910 that will be used for the treatment. The doctor then identifies a number of pressure points 920 on the brace that are applying forces on the patient's body and attaches a sensor for each pressure point 930. The sensors are attached to the back brace with adhesive material (e.g. a double-sided self-adhesive tape, hot glue, etc.) on one of the large flat faces of the sensor. On the opposite to the back brace flat surface of the pressure sensors, the doctor attaches a foam strip (e.g. of polyethylene or other synthetic or natural material) for avoiding irritations of the patient's skin and for increased comfort. The doctor connects the sensors 940 with cables to the connectors of the wearable device (or wirelessly in an alternative exemplary embodiment) and then the doctor checks if the LED indicators on the wearable device are lit 960. If not, the doctor replaces a pre-installed battery 980 and connects the battery 985 to the wearable device (e.g., an off-the-shelf battery or a rechargeable battery). In another aspect, the device has no pre-installed battery and the doctor needs to install a battery during the first time the wearable device is used. If the wearable device has Light Emitting Diode (LED) indicators lit 960, the doctor attaches the wearable device 970 on the external side of the brace with adhesive material, or with Velcro®, or at the preexisting Velcro® straps that are used for tightening and loosening the brace. In an alternative exemplary implementation, the doctor attaches the wearable device 970 in a cavity (e.g., such as is denoted at 111 in FIG. 1B) in the material of the brace with adhesive material, or with Velcro®, etc.

Wearable Device Connection with the Patient's Mobile Application

FIG. 10 shows a flow diagram of connecting the wearable device with a mobile application. Flowchart 1000 starts with the patient's mobile application prompting the patient to connect with the wearable device via either a unique Quick Response (QR) code or via a Bluetooth scan (1010) and checks if the patient chose the QR code scan option (1015). If the patient has chosen the QR scan option, the (primary or secondary) camera of the patient's mobile device (or any computing apparatus he uses) scans for a unique QR code (1020). The patient identifies a QR tag containing the unique QR code (that is given or messaged to him by the doctor, or which is attached on or inside the case of the wearable device) and scans (1030) it by pointing the camera of his mobile device (or any computing apparatus he uses) towards the QR tag. The mobile device starts a Bluetooth scanning process using information acquired from the QR code (1040).

If the patient chose the Bluetooth scan option, the mobile device scans all active Bluetooth devices in the area (1017) and prompts the patient to choose the device based on its Bluetooth name (1019).

If the wearable device is discoverable from the mobile application running on the patient's mobile device (1050), then the mobile application stores the connection information to its local memory (e.g. a flash or other type of memory which stores data in any known data format in an optional database) for future connections (1060), and uploads connection information associated with the patient and his wearable device to the server (1070), before ending connecting the wearable device with the mobile application.

If the wearable device is not discoverable from the mobile application (1050), the mobile application requests the patient to reset the wearable device via pressing a reset button in the wearable device (1052). If Light Emitting Diode (LED) indications light on the mobile device (1054), the methodology branches to step 1010, else the doctor replaces the wearable device (1056) and then step 1010 is executed.

System Calibration

FIG. 11 shows a flow diagram of calibrating the system. Methodology 1100 starts with the patient visiting his doctor for examination (1105) during an appointment. The doctor tightens the straps of the back brace (1110) and adjusts the back brace to the proper position (1115) on the patient's torso, exerting a desired pressure on the patient's spine via preselected pressure points. The doctor initializes the calibration process through a doctor's web application (1120) running at the server, and the mobile application running on the patient's mobile device retrieves the Bluetooth connection information (1125) of the wearable device by connecting to the server. The mobile application on the patient's mobile device scans for the wearable device using the stored connection information (1030).

If the wearable device is not discoverable (1135), the mobile application on the patient's mobile device increments a counter (1190) (which was already in reset state) and repeatedly scans 1130 (e.g., for five more times) for the wearable device until a threshold number of unsuccessful scans is reached 1195 and then the mobile application ends the process.

If the wearable device is discoverable (1135), the mobile application requests the pressure data from the wearable device using a Bluetooth connection (1140). The data are retrieved through a Bluetooth communication protocol (1145), transferred/broadcasted to the mobile application (1150), stored inside the mobile application (1160) and at the mobile device's memory, calibration results are sent to the wearable device (1165), and are then uploaded to the server (1170). Then the results of the calibration are displayed to the mobile application of the patient's mobile device (1180) (or any other computing apparatus used by the patient) and in the web application accessed by the doctor (1185) using his computing device, and the process ends.

System Adjustment

FIG. 12 shows a flow diagram of adjusting the system. Flowchart 1200 starts with the patient wearing the brace (1205) and starting an adjustment functionality inside the mobile application running in his mobile device (or any other computing apparatus) (1210). The mobile application retrieves the calibration values that are stored in the server (1215). The calibration values are the pressure values that the doctor suggested to the patient during the Calibration process (please refer to FIG. 11). The mobile application retrieves the Bluetooth connection information of the wearable device (1220) from the mobile device's memory and scans using a Bluetooth protocol for the wearable device using the stored connection information (1225).

If the wearable device is not discoverable (1230), the mobile application increments a counter (1235) (which was in reset state at the beginning of process 1200) and repeatedly scans (1225) until a threshold is reached (1240) (e.g. for five more times) before ending the process.

If the wearable device is discoverable (1230), the mobile application requests the pressure data from the wearable device using the Bluetooth connection (1245) and it displays the pressure values live on the screen of the mobile app (1250). The patient then gradually tightens the straps of the back brace based on the indications of the application (1255). If the values of the pressure sensor readings have not reached the calibration values from the tightening of the straps (1260), and the straps have not been tightened to the prescribed points (1290), the mobile application requests pressure data from the wearable device using the Bluetooth connection (1245) and checks if the straps have been tightened to the end (1280) If the straps have been tightened to the end (1280), it is indicated to change the back brace (1285) and methodology 1200 ends. If the straps have not been tightened to the end (1280), step 1245 is executed. If the straps have been tightened to the prescribed points (1290), an indication is given to correct (1292) the back brace and to book a new appointment with the doctor (1294), and the methodology ends.

If the pressure values have reached the calibration values from the tightening of the straps (1260), adjustment results are stored in the mobile application 1260, are uploaded to the server (1270), and the methodology ends.

Patient Access to Data

The patient can use an application running on his smartphone (or at any computing device he uses) to access the metrics from the server, instruction on how to efficiently use the brace, advice from the doctor for correcting the application of the brace on the patient's torso, changes in the treatment regime, and a gamification of the treatment for incentivizing him to follow the treatment.

In a first aspect, the patient's application motivates young adolescents to increase compliance in the prescribed use of the brace through actual wearing metrics, achievements and badges. The patient's application also provides analytics (e.g. charts, graphs, etc.) for the patient's daily, weekly, monthly and yearly activity. This is complemented by instructions on the correct use of the brace, advice from his doctor relating the treatment, and reminders of scheduled appointments.

In a second aspect, the patient's application uses gamification, which is particularly suitable for children of pre-adolescent age. This aspect, the patient's application presents to the child patient a game, which is adapted to the patient's treatment regime and progress (as provided by the usage metrics) and uses awards related to the correct personalized use of the brace.

An example game consists of an incremental game where the patient can oversee an ant hive. The purpose of the game is to expand the ant hive by purchasing ants. The game has two currencies: “leaves” and “strawberries”. The ants collect leaves. The player can trade them in order to purchase more ants for the hive. Furthermore, the player can collect more leaves by tapping the screen. The hive has an upper limit in the ant population at a specific level. The user can upgrade the hive level using the second currency (strawberries). The only way the user can collect strawberries is by wearing its back brace for the prescribed time. Furthermore, the app has a set of achievements and awards in order to increase the wearing time of the brace. Other games can be created and presented to child patients for selecting their preferred one and further boost their incentivization. Other game scenarios can be accommodated without limiting the scope of protection of the invention.

Doctor Access to Data

The doctor can create on the server (using his computing device) a patient account and a personalized treatment regime, and access from the server the metrics, adapt the treatment regime, send advice to the patient for correcting the application of the brace, schedule appointments with the patient at optimal times and dates that are beneficial for managing the progress of the therapy based on the metrics, and receive alerts associated with the metrics and the patient's progress.

The above actions and related data may optionally be shared with other software and systems used by the doctor (e.g. calendar applications, Patient Archiving and Communication System (PACS), Patient Information Folder (PIF), Hospital Information System (HIS), etc.).

Software Architecture

The system is based on following software modules:
Embedded software running at the wearable device for collecting, pre-processing (i.e. at least digitizing and time-stamping), and forwarding to the patient application on the patient computing device the pressure measurements in real time or whenever a connection between the wearable device embedded software and the patient application is established.
A patient application for motivating the guiding the patient 24/7 to correctly wear the brace and attend appointments with his doctor. It runs on the patient's computing device, typically a smart phone which uses connects to the server to access the processed data to present to the patient.
A doctor application for having 24/7 access to the metrics of the brace usage, progress of the therapy, and manage the patient and the therapy. It runs on the server and is accessed by the doctor's computing device.
A server software package for storing sensory data, analyzing and sharing metrics and other processed data with the patient and doctor's applications, and enforcing authorization, authentication of users, and authentication and encryption of data stored and exchanges to the other software used by the patient and his doctor, as well as external systems and applications.

The embedded software running at the wearable device performs at least one of the following:
stores the at least one pressure measurement, the at least one sensor identifier, and the at least one time-date data field to the second memory module;
associates the at least one pressure measurement, the at least one sensor identifier, and the at least one time-date data field with the patient;
transmits the at least one pressure measurement, the at least one sensor identifier, and the at least one time-date data field associated with the patient to a server;
receives from the server (via the patient application) and executes a second set of instructions for performing at least one of presenting in real time the at least one pressure measurement together with a description of the associated pressure sensor, indicating adjustments to the back brace for modifying at least one of the least one pressure measurement, presenting metrics on time use and pressure measurements of the back brace device, presenting benefits, associated with the metrics, to the patient, presenting target accomplishments associated with the metrics, gamifying a use of the back brace by presenting a game using the metrics, associating the game with at least one award, and presenting physical exercises, prescribed by the doctor for the spine deformation treatment. Other functions may also be performed, as is apparent to any person skilled in the art.

The patient application performs at least one of the following:
stores the at least one pressure measurement, the at least one sensor identifier, and the at least one time-date data field to the second memory module;
associates the at least one pressure measurement, the at least one sensor identifier, and the at least one time-date data field with the patient;
transmits the at least one pressure measurement, the at least one sensor identifier, and the at least one time-date data field associated with the patient to a server;
receives from the server and execute a second set of instructions for performing at least one of presenting in real time the at least one pressure measurement together with a description of the associated pressure sensor, indicating adjustments to the back brace for modifying at least one of the least one pressure measurement, presenting metrics on time use and pressure measurements of the back brace device, presenting benefits, associated with the metrics, to the patient, presenting target accomplishments associated with the metrics, gamifying a use of the back brace by presenting a game using the metrics, associating the game with at least one award, and presenting physical exercises, prescribed by the doctor for the spine deformation treatment.

The server software performs at least one of the following:
manages a plurality of patient accounts, each associated with one of the plurality of patients;
creates a plurality of personalized spine deformation treatments, each associated with one or more patient accounts, the back brace is associated with the one of the plurality of patients, and the pressure sensors are associated with the back brace which is associated with the patient;
receives from the computing apparatus the at least one pressure measurement, the at least one sensor identifier, and the at least one time-date data field associated with the patient;
associates the pressure measurements, the sensor identifier, and the time-date data field with the patient accounts;
creates metrics on time use and pressure measurements received from the computing apparatus;
associates benefits and target accomplishments with the metrics;
gamifies the use of the back brace by using the metrics;
associates the gamifying the use of the back brace with one or more awards;
prescribes physical exercises for the patient for the spine deformation treatment;
manages appointments, associated with patient accounts.

Other functions may also be performed by the software, as is apparent to any person skilled in the art.

The patient and doctor software applications may be written in any computer language (high-level, descriptive—e.g. eXtensible Markup Language (XML), low-low level, assembly language, etc.) or combination of computer languages.

The server software may be written in any computer language or combination of computer languages and be offered as a web application, or as web services.

The patient's application and the server software are designed to inform and guide the patient in real time, and also to incentivize the patient to more efficiently wear the back brace.

The doctor's application and the server software are designed to allow the doctor to more efficiently monitor, intervene, and manage any number of patients and their treatments in real time.

Hardware Architecture

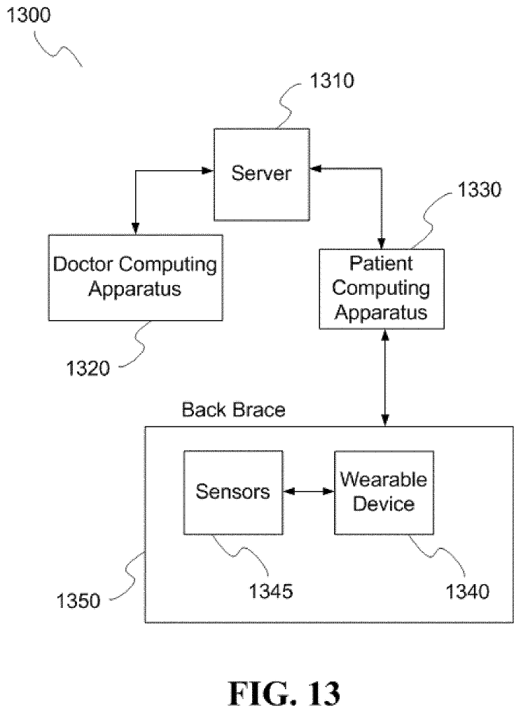

FIG. 13 shows a schematic diagram of the interaction of the system hardware. System 1300 consists of a server 1310, e.g., a cloud or application server, connected with a doctor's computing apparatus 1320, e.g., a tablet, and a patient's computing apparatus 1330, e.g., a smartphone. The patient's computing apparatus 1330 is connected with a wearable device 1340, collecting pressure sensor measurements from one or more sensors 1345. The connections are either wired or wireless, or a combination of the two. The connection of wearable device 1340 with patient apparatus 1330 is, for example, via a Bluetooth Low Energy (Bluetooth LE) wireless link. Wearable device 1340 and sensors 1345 are attached (i.e. installed) at the back brace denoted here with numeral 1350.

In an alternative exemplary embodiment, system 1300 consists only of wearable device 1340 and the patient's computing apparatus 1330, and server 1310 and doctor's computing apparatus 1320 are external modules or systems.

It will be appreciated that the present invention solves the problem of facilitating doctors in efficiently monitoring and managing treatment of spinal deformations, while aiding and incentivizing patients to efficiently follow the prescribed treatment regime. It solves the problem using real-time pressure measurements, data analysis, and scheduling of doctor interventions and appointments with the patient for optimizing the personalized treatment of the patient's spinal deformation. As described herein according to the embodiments, a wearable device and pressure sensors are attached to the inside of a back brace, for collecting, timestamping and sending pressure measurements to a server, using a patient computing apparatus as a relay. The server creates metrics, and manages treatment, giving access to processed data to the patient's doctor and to the patient, and offering gamification for patient incentivization to the use of the brace according to the treatment regime prescribed by the doctor. Processed data created by the server are presented to the patient and doctor computing devices.

The present innovative solution can also be implemented by software written in any programming language, or in an abstract language (e.g., a metadata-based description which is then interpreted by a software or hardware component). The software running in the above-mentioned hardware, effectively transforms a general-purpose or a special-purpose hardware or computing device, apparatus or system into one that specifically implements the present innovative solution. In another aspect an embedded system is used for the wearable device.

Alternatively, the present innovative solution can be implemented in Application Specific Integrated Circuits (ASIC) or other hardware technology.

The above exemplary embodiment descriptions are simplified and do not include hardware and software elements that are used in the embodiments but are not part of the current invention, are not needed for the understanding of the embodiments, and are obvious to any user of ordinary skill in related art. Furthermore, variations of the described system architecture are possible, where, for instance, some servers may be omitted or others added.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

In one or more exemplary embodiments, the functions described may be implemented in hardware, software, firmware, or any combination thereof. If implemented in software, the functions may be stored on or transmitted over as one or more instructions or code on a computer readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A system for use with a back brace configured to be worn by a patient for efficient management of spine deformation treatments, comprising:
at least one pressure sensor configured to be attached to the back brace at an area of contact containing a point of pressure to be applied to a corresponding area of the patient's torso for correcting a spinal deformation and/or constraining the spine;
a battery module;
a real-time clock module;
a first memory module;
a first communications module; and
a first processor connected with the at least one pressure sensor, the battery module, the real-time clock module, the first memory module, and the first communications module, the processor running a first set of instructions causing the processor to:
receive at least one pressure measurement from the at least one pressure sensor in real time;
associate each of the at least one pressure measurement with a sensor identifier of one of the at least one pressure sensors;
receive at least one time-date data field from the real-time clock module;
associate each of the at least one pressure measurement with one of the at least one time-date data field and with a pressure sensor identifier;
save the at least one pressure measurement, the at least one pressure sensor identifier, and the at least one time-date data field to the first memory module;
connect to a patient computing apparatus; and
transmit the at least one pressure measurement, the at least one pressure sensor identifier, and the at least one time-date data field to the patient computing apparatus for (a) informing and guiding in real time, and incentivizing a patient to more efficiently wear the back brace, and (b) enabling a doctor to more efficiently monitor, intervene, and manage a plurality of patients and their treatments in real time.

2. The system of claim 1, wherein the battery module, the real-time clock module, the first memory module, the first communications module, and the first processor are configured as a wearable device.

3. The system of claim 1, further comprising a patient computing device, wherein the computing device comprises a second memory module and a second processor, wherein the second processor runs a software application configured to perform at least one of:
store the at least one pressure measurement, the at least one sensor identifier, and the at least one time-date data field to the second memory module;
associate the at least one pressure measurement, the at least one sensor identifier, and the at least one time-date data field with the patient;
transmit the at least one pressure measurement, the at least one sensor identifier, and the at least one time-date data field associated with the patient to a server; and
receive from the server and execute a second set of instructions for performing at least one of:
presenting in real time the at least one pressure measurement together with a description of the associated pressure sensor;
indicating adjustments to the back brace for modifying at least one of the least one pressure measurement;
presenting metrics on time use and pressure measurements of the back brace device;
presenting benefits, associated with the metrics, to the patient;
presenting target accomplishments associated with the metrics;
gamifying a use of the back brace by presenting a game using the metrics;
associating the game with at least one award; and
presenting physical exercises, prescribed by the doctor for the spine deformation treatment.

4. The system of claim 3, wherein the patient computing apparatus is selected from a set comprising a smart phone, a smart watch, and a computing device.

5. The system of claim 3, further comprising a server, wherein the server comprises a third memory module and a third processor, wherein the third processor runs software configured to allow a doctor to perform at least one of:
manage a plurality of patient accounts, each associated with one of the plurality of patients;
create a plurality of personalized spine deformation treatments, each associated with (a) one of the plurality of patient accounts, (b) the back brace associated with the one of the plurality of patients, and (c) the at least one pressure sensor associated with the back brace associated with the one of the plurality of patients;
receive from the patient computing apparatus the at least one pressure measurement, the at least one sensor identifier, and the at least one time-date data field associated with the patient;
associate the at least one pressure measurement, the at least one sensor identifier, and the at least one time-date data field with one of the plurality of patient accounts;
create metrics on time use and pressure measurements received from the patient computing apparatus;
associate (i) benefits, and (ii) target accomplishments with the metrics;
gamify a use of the back brace by using the metrics;
associate the gamifying the use of the back brace with at least one award;
prescribe physical exercises for the patient for the spine deformation treatment; and
manage a plurality of appointments, each associated with one of the plurality of patient accounts.

6. The system of claim 5, wherein the managing of the plurality of appointments is done by the third processor by quantifying the progress of each of the plurality of patients.

7. The system of claim 6, wherein the quantifying of the progress of each of the plurality of patients is based on the metrics and target accomplishments associated with each of the plurality of patients.

8. The system of claim 6, wherein the management of the plurality of appointments is based on determining the outcome of the treatment associated with each of the plurality of patients before fixing the appointment.

9. The system of claim 1, wherein the first communications module is a Bluetooth Low Energy transceiver.

10. The system of claim 1, wherein the at least one pressure sensor is:
selected from resistive and capacitive pressure sensors;
attached to the brace, at a point of pressure between the brace and the patient's body, with an adhesive material applied to the bottom side of the at least one pressure sensor;
covered with a synthetic foam, wherein the synthetic foam is attached to the at least one pressure sensor with the adhesive material applied to the upper side of the at least one pressure sensor; and
connected to the processor with connection means selected from cables and wireless links.

11. The system of claim 2, wherein the wearable device is carried by means selected from (a) attaching the wearable device to an external side of the brace, (b) carrying the wearable device inside a pocket of a garment worn by the patient, (c) wearing the wearable device using a strap, and (d) attaching the wearable device in a cavity inside a material of the back brace.

12. A method for efficient management of spine deformation treatments, the method being executed at a processor of a wearable device and comprising the steps of:
receiving at least one pressure measurement from at least one pressure sensor in real time;
associating each of the at least one pressure measurement with a sensor identifier of one of the at least one pressure sensors;
receiving at least one time-date data field from a real-time clock module;
associating each of the at least one pressure measurement with one of the at least one time-date data field and with a pressure sensor identifier;
saving the at least one pressure measurement, the at least one pressure sensor identifier, and the at least one time-date data field to a first memory module;
connecting to a patient computing apparatus; and
transmitting the at least one pressure measurement, the at least one pressure sensor identifier, and the at least one time-date data field to the patient computing apparatus for (a) informing and guiding in real time, and incentivizing a patient to more efficiently wear a back brace, and (b) enabling a doctor to more efficiently monitor, intervene, and manage a plurality of patients and their treatments in real time.

13. The method of claim 12, further comprising executing on a patient computing device the steps of:
storing the at least one pressure measurement, the at least one sensor identifier, and the at least one time-date data field to the second memory module;
associating the at least one pressure measurement, the at least one sensor identifier, and the at least one time-date data field with the patient;
transmitting the at least one pressure measurement, the at least one sensor identifier, and the at least one time-date data field associated with the patient to a server;
receiving from the server and executing a second set of instructions for performing at least one of:
presenting in real time the at least one pressure measurement together with a description of the associated pressure sensor;
indicating adjustments to the back brace for modifying at least one of the least one pressure measurement;
presenting metrics on time use and pressure measurements of the back brace device;
presenting benefits, associated with the metrics, to the patient;
presenting target accomplishments associated with the metrics;
gamifying a use of the back brace by presenting a game using the metrics;
associating the game with at least one award; and
presenting physical exercises, prescribed by the doctor for the spine deformation treatment.

14. The method of claim 13, further comprising executing on a server the steps of:
managing a plurality of patient accounts, each associated with one of the plurality of patients;
creating a plurality of personalized spine deformation treatments, each associated with (a) one of the plurality of patient accounts, (b) the back brace associated with the one of the plurality of patients, and (c) the at least one pressure sensor associated with the back brace associated with the one of the plurality of patients;
receiving from the patient computing apparatus the at least one pressure measurement, the at least one sensor identifier, and the at least one time-date data field associated with the patient;
associating the at least one pressure measurement, the at least one sensor identifier, and the at least one time-date data field with one of the plurality of patient accounts;
creating metrics on time use and pressure measurements received from the patient computing apparatus;
associating (i) benefits, and (ii) target accomplishments with the metrics;
gamifying a use of the back brace by using the metrics;
associating the gamifying the use of the back brace with at least one award;
prescribing physical exercises for the patient for the spine deformation treatment; and
managing a plurality of appointments, each associated with one of the plurality of patient accounts.

15. The method of claim 14, wherein:
the managing of the plurality of appointments is done by quantifying the progress of each of the plurality of patients;
the quantifying of the progress of each of the plurality of patients is based on the metrics and target accomplishments associated with each of the plurality of patients; and
the management of the plurality of appointments is based on determining the outcome of the treatment associated with each of the plurality of patients before fixing the appointment.

16. A non-transitory computer program product for efficient management of spine deformation treatments, comprising instruction configured to cause a processor of a wearable device to:
receive at least one pressure measurement from at least one pressure sensor in real time;
associate each of the at least one pressure measurement with a sensor identifier one of the at least one pressure sensors;
receive at least one time-date data field from a real-time clock module;
associate each of the at least one pressure measurement with one of the at least one time-date data field and with a pressure sensor identifier;
save the at least one pressure measurement, the at least one pressure sensor identifier, and the at least one time-date data field to a first memory module;
connect to a patient computing apparatus; and
transmit the at least one pressure measurement, the at least one pressure sensor identifier, and the at least one time-date data field to the patient computing apparatus for (a) informing and guiding in real time, and incentivizing a patient to more efficiently wear a back brace, and (b) enabling a doctor to more efficiently monitor, intervene, and manage a plurality of patients and their treatments in real time.

17. The non-transitory computer program product of claim 16, further comprising instructions to cause a patient computing device to:
store the at least one pressure measurement, the at least one sensor identifier, and the at least one time-date data field to the second memory module;
associate the at least one pressure measurement, the at least one sensor identifier, and the at least one time-date data field with the patient;
transmit the at least one pressure measurement, the at least one sensor identifier, and the at least one time-date data field associated with the patient to a server;
receiving from the server and executing a second set of instructions for performing at least one of:
present in real time the at least one pressure measurement together with a description of the associated pressure sensor;
indicate adjustments to the back brace for modifying at least one of the least one pressure measurement;
present metrics on time use and pressure measurements of the back brace device;
present benefits, associated with the metrics, to the patient;
present target accomplishments associated with the metrics;
gamify a use of the back brace by presenting a game using the metrics;
associate the game with at least one award; and
present physical exercises, prescribed by the doctor for the spine deformation treatment.

18. The non-transitory computer program product of claim 17, further comprising instructions to cause a server to:
manage a plurality of patient accounts, each associated with one of the plurality of patients;
create a plurality of personalized spine deformation treatments, each associated with (a) one of the plurality of patient accounts, (b) the back brace associated with the one of the plurality of patients, and (c) the at least one pressure sensor associated with the back brace associated with the one of the plurality of patients;
receive from the patient computing apparatus the at least one pressure measurement, the at least one sensor identifier, and the at least one time-date data field associated with the patient;
associate the at least one pressure measurement, the at least one sensor identifier, and the at least one time-date data field with one of the plurality of patient accounts;
create metrics on time use and pressure measurements received from the patient computing apparatus;
associate (i) benefits, and (ii) target accomplishments with the metrics;
gamify a use of the back brace by using the metrics;
associate the gamifying the use of the back brace with at least one award;
prescribe physical exercises for the patient for the spine deformation treatment; and
manage a plurality of appointments, each associated with one of the plurality of patient accounts.

19. The non-transitory computer program product of claim 18, wherein:
the managing of the plurality of appointments is done by quantifying the progress of each of the plurality of patients;
the quantifying of the progress of each of the plurality of patients is based on the metrics and target accomplishments associated with each of the plurality of patients; and
the management of the plurality of appointments is based on determining the outcome of the treatment associated with each of the plurality of patients before fixing the appointment.

20. A back brace configured to be worn by a patient for treating spinal deformations, the back brace comprising:
at least one pressure sensor configured to be attached to the back brace at a point of pressure with the body of a patient;
at least one strap configured to be tightened for adjusting a pressure at the point of pressure; and
a wearable device configured (a) for being attached to one of an outside of the back brace and inside a cavity formed in the back brace, (b) for processing signals from the at least one pressure sensor, (c) for communicating with at least one external computing apparatus, (d) for informing and guiding in real time and incentivizing a patient to more efficiently wear the back brace by at least providing indications to the patient for tightening the at least one strap by comparing a pressure value provided by the at least one pressure sensor with at least one calibration value, and (e) for enabling a doctor to more efficiently monitor, intervene, and manage the patient and treatment in real time.
